(19) United States
(12) Patent Application Publication
(10) Pub. No.: US 2026/0176223 A1
(43) Pub. Date: Jun. 25, 2026

(54) Isomerization of trans-1,2-difluoroethylene (HFO-1132(E)) and/or cis-1,2-difluoroethylene (HFO-1132(Z))

(71) Applicant: Solstice Advanced Materials US, Inc., Morris Plains, NJ (US)

(72) Inventors: Akbar Mahdavi-Shakib, Buffalo, NY (US); Haiyou Wang, Amherst, NY (US); Terris Yang, East Amherst, NY (US); Daniel C. Merkel, Orchard Park, NY (US); Xinrui Yu, Furlong, PA (US)

(21) Appl. No.: 19/402,057

(22) Filed: Nov. 26, 2025

Related U.S. Application Data
(60) Provisional application No. 63/736,420, filed on Dec. 19, 2024.

(51) Int. Cl. C07C 17/358; B01J 19/00; B01J 19/24

(52) U.S. Cl. CPC C07C 17/358; B01J 19/0013; B01J 19/2415; B01J 2219/00135; B01J 2219/185

(57) ABSTRACT

Production of HFO-1132 and, in particular, HFO-1132E, may be produced from 1,1,2-trichloro-1,2,2-trifluoroethane (CFC-113) or CTFE. The present disclosure provides a non-catalytic method for producing trans-1,2-difluoroethylene (HFO-1132(E)), comprising: providing a reactant composition comprising cis-1,2-difluoroethylene (HFO-1132(Z)); and isomerizing the HFO-1132(Z) in an electric heater reactor or a tubular reactor at a temperature of from about 350° C. to about 600° C. to produce a product composition comprising HFO-1132(E).

### CROSS-REFERENCE TO RELATED APPLICATION

This application claims priority to Provisional Application No. 63/736,420, filed Dec. 19, 2024, which is herein incorporated by reference in its entirety.

### FIELD

The present disclosure is directed to methods for producing 1,2-difluoroethylene (HFO-1132), including trans-1,2-difluoroethylene (HFO-1132(E)) and/or cis-1,2-difluoroethylene (HFO-1132(Z)) and, in particular, to methods of conducting isomerization reactions between trans-1,2-difluoroethylene (HFO-1132(E)) and/or cis-1,2-difluoroethylene (HFO-1132(Z)).

### BACKGROUND

1,2-difluoroethylene (HFO-1132) has recently found increased utility for a variety of uses. HFO-1132 may exist as a mixture of two geometric isomers, the (E) or trans isomer and the (Z) or cis isomer, which may be used separately or together in various proportions. Potential end use applications of HFO-1132 include refrigerants, either used alone or in blends with other components, solvents for organic materials, and as a chemical intermediate in the synthesis of other halogenated hydrocarbon solvents.

Improved methods for the production of HFO-1132 and, in particular, HFO-1132(E), are desired.

### SUMMARY

The present disclosure provides methods of isomerizing cis-1,2-difluoroethylene (HFO-1132(Z)) and trans-1,2-difluoroethylene (HFO-1132(E)) through a non-catalyzed pathway. Advantageously, the isomerization reactions through a non-catalyzed pathway show high selectivity for the desired product with minimal by-products.

In one form thereof, the present disclosure provides a method for producing trans-1,2-difluoroethylene (HFO-1132(E)), comprising: providing a reactant composition comprising cis-1,2-difluoroethylene (HFO-1132(Z)); and isomerizing the HFO-1132(Z) in an electric heater reactor at a temperature of from about 325° C. to about 600° C. to produce a product comprising HFO-1132(E).

In another form thereof, the present disclosure provides a method for producing trans-1,2-difluoroethylene (HFO-1132(E)), comprising: providing a reactant composition comprising cis-1,2-difluoroethylene (HFO-1132(Z)); and isomerizing the HFO-1132(Z) in a tubular reactor at a temperature of from about 325° C. to about 600° C. to produce a product composition comprising HFO-1132(E).

In another form thereof, the present disclosure provides an integrated system for the production of HFO-1132(E), the system comprising: an electric heater reactor comprising an electric heating element configured to maintain a reactor temperature from about 325° C. to about 600° C. and to heat at least part of an reactant composition; the electric heater reactor configured to produce a product stream comprising HFO-1132(E); and a separator fluidly coupled to the product stream, the separator configured to produce a concentrated product stream and a recycle stream, the concentrated product stream including a concentration of HFO-1132(E) that is greater than a concentration of HFO-1132(Z) in the reactant composition.

### BRIEF DESCRIPTION OF THE DRAWINGS

FIG. 1a is a schematic diagram of an apparatus used in a once-through process for the conversion of HFO-1132(Z) to HFO-1132(E).

FIG. 1b is a schematic diagram of the side view of an electric heater reactor for use in a process of, for example, the conversion of HFO-1132(Z) to HFO-1132(E).

FIGS. 2a, 2b, 2c, and 2d are schematic diagrams of apparatuses used in integrated processes for the conversion of HFO-1132(Z) to HFO-1132(E).

FIGS. 3a and 3b are schematic diagrams of apparatuses used in integrated processes for the conversion of HFO-1132(Z) to HFO-1132(E).

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

As used herein, the phrase “based on total moles of organic components of the composition” refers only to carbon-containing components and does not include or encompass non-carbon-containing components such as hydrogen (H₂) or hydrogen chloride (HCl).

As used herein, the phrase “light impurities” refer to molecules that have lower boiling points than HFO-1132(E) or HFO-1132(Z).

As used herein, the phrase “heavy impurities” refer to molecules that have higher boiling points than HFO-1132(E) or HFO-1132(Z).

As used herein, the phrase “once-through process” refers to a process during which a reactant composition is conveyed via a single pass through a reactor to form a product composition without further processing (e.g., going through a separation column or recycling).

As used herein, the phrase “once-through product” refers to a product composition collected and analyzed at the end of a once-through process.

As used herein, the phrase “integrated process” refers to a process during which a once-through product composition is then further processed in an integrated system, for example, going through a separation column for purification and/or recycling.

As used herein, the phrase “end product” refers to a product composition collected and analyzed at the end of an integrated process.

As used herein, the overall yield of a target product is calculated using the following equation:

$$Overall yield = \frac{Target product}{Initial reactant} \times 100 \%$$

As used herein, conversion of a reactant molecule (molecule X) during a reaction is calculated using the following equation:

$$\% conversion of molecule X = ( 100 - molecule X mol . \% in the organic components of a product mixture )$$

### II. Overview

The present disclosure provides a method for producing trans-HFO-1132(E) from cis-HFO-1132(Z) (“Process 1”), which includes providing a feed stream (e.g., reactant composition) containing substantially pure HFO-1132(Z) or a mixture of HFO-1132(E) and HFO-1132(Z), converting at least a portion of HFO-1132(Z) to HFO-1132(E), and separating and recovering HFO-1132(E) or a mixture of HFO-1132(E) and HFO-1132(Z). The reactor may be an electric heater reactor without use of a catalyst, or a tubular reactor without use of a catalyst.

Schematic equation for Process 1 is represented below:

### Process 1

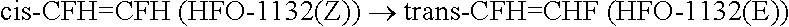

The feed stream of a mixture of HFO-1132(E) and HFO-1132(Z) may be obtained via various methods and starting materials. For example, a mixture of HFO-1132(E) and HFO-1132(Z) may be obtained using CFCl₂—CF₂Cl (CFC-113) as a starting material via a two-step reaction process summarized below:

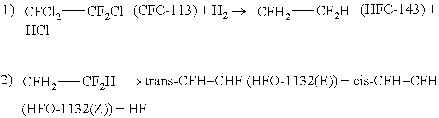

A mixture of HFO-1132(E) and HFO-1132(Z) may also be obtained using chlorotrifluoroethylene (CTFE) or a mixture of CTFE and CF₂═CHF (HFO-1123) as starting materials via a two-step reaction process summarized below:

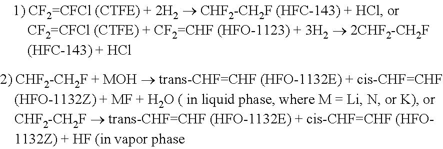

Further details regarding each of the isomerization reactions of Process 1 are set forth below.

### III. Process 1

### General Process 1

As discussed above, a feed stream may include 1,2-difluoroethylene (HFO-1132) obtained via various methods or starting materials and including a mixture containing either substantially pure cis-HFO-1132(Z), or both the trans-HFO-1132(E) and cis-HFO-1132(Z) isomers.

In an integrated process, the cis-1,2-difluoroethylene HFO-1132(Z) isomer may be converted to the trans-1,2-difluoroethylene (HFO-1132(E)) isomer by exposure to heat without use of a catalyst and further purified to form an end product comprising, consisting essentially of, or consisting of, the trans-1,2-difluoroethylene (HFO-1132(E)) isomer in high purity, such as at least about 95 wt. %, at least about 99.0 wt. %, at least about 99.9 wt. %, at least about 99.99 wt. % or greater, and for each of the foregoing, less than or equal to 100%, or within any range encompassed by two of the foregoing values as endpoints, based on total moles of the organic components of the end product composition.

In a once-through process, the isomerization reaction in Process 1 is controlled by thermodynamics (e.g., temperature under which the reaction is conducted) independent of pressure. As such, the conversion of HFO-1132(Z) to HFO-1132(E) in a once-through process reaches its maximum value once the isomerization reaction reaches its thermodynamic equilibrium. The % conversion of HFO-1132(Z) in a once-through process starting with a reactant composition including at least 95% and less than or equal to 99.9% HFO-1132(Z) may be at least about 8%, at least about 16%, at least about 20%, at least about 25%, at least about 30%, at least about 35% or greater, and for each of the foregoing, less than or equal to 40%, or within any range encompassed by two of the foregoing values as endpoints, based on total moles of the organic components of the once-through product composition.

The isomerization reaction may be conducted in a suitable reaction vessel or reactor. The reactor is preferably constructed from materials which are resistant to corrosion such as nickel and its alloys, including Hastelloy (for example, Hastelloy C276), Inconel (for example Inconel 600), and Monel. The reactor may be made to include materials that may serve a similar function as a catalyst, but for a non-catalytic reaction.

The reactor may be first cleaned and flushed with an inert gas such as nitrogen. Products may be flowed through one or more scrubbers to remove byproducts contained in the feed stream from the stream, such as hydrogen fluoride (HF) and/or hydrogen chloride (HCl), and the reaction products may be collected by capture in a cooled cylinder, for example.

### Suitable Reactors

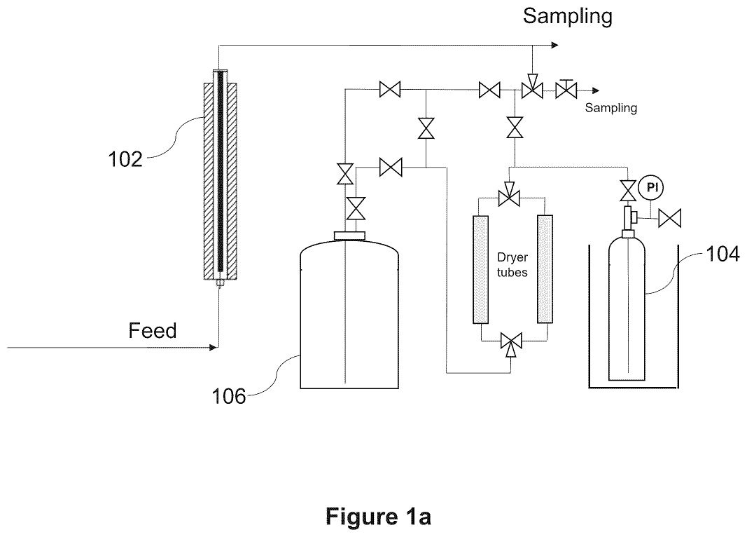

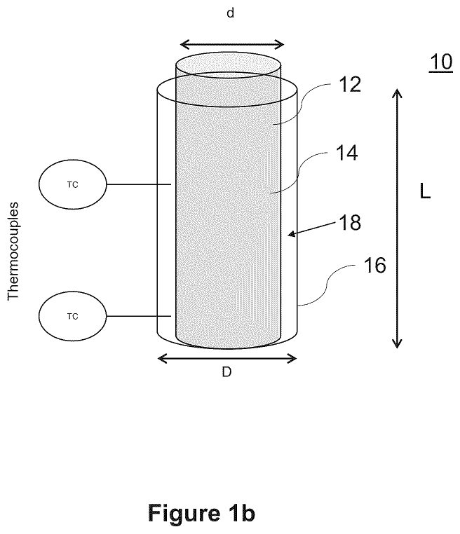

Schematic diagrams of various process flows illustrating suitable components for the reaction in Process 1 using an electric heater reactor or a tubular reactor are provided in FIGS. 1-3. The reactor may be an electric heater reactor with at least one electric heating element including a tube (e.g., a sheath), a metal alloy wire such as Nichrome wire, and a compacted metal oxide material (e.g., MgO) to heat the reactor tube to a desired outer surface temperature effective for the reaction to take place. The electric heating element may be packed with MgO powder to conduct heat from Nichrome wire while also acting as an electrical insulator. The manufacturing process includes inserting Nichrome wire into a tube (e.g., a sheath), packing with MgO powder, and rolling the tube again to compact the MgO and form a ceramic-like material. The MgO powder is isolated from the process because of the sheath tube wall throughout the interior of the heater. The sheath tubes may be constructed from a material which is resistant to temperature and/or corrosive effects of HF or HCl.

FIG. 1b is a schematic diagram of the side view of an electric heater reactor for use in a process of, for example, the conversion of HFO-1132(Z) to HFO-1132(E). As shown, the electric heater reactor 10 includes an electric heating element 12. The electric heating element 12 may include a sheath 14 and additional electric heating elements (e.g., a metal wire, a compacted metal oxide material) packed inside the sheath 14. As shown, the electric heating element 12 having a length “L” is placed inside a reactor tube 16. The outer diameter “d” of the electric heating element 12 is smaller than the inner diameter “D” of the reactor tube 16, forming an empty space 18 in between the outer surface of the electric heating element 12 and the inner surface of the reactor tube 16. The volume of the space 18 is determined by the length “L” of the electric heating element 12 encompassed by the reactor tube 16 and the difference between the outer diameter “d” of the electric heating element 12 and the inner diameter “D” of the reactor tube 16. During a reaction process (e.g., process 1), a reactant composition is fed into the space 18 of the electric heater reactor 10 and heated by the electric heating element 12. The reactant composition contacts the sheath 14 or the outer surface of the electric heating element 12 to be heated during the process.

Suitable reactors for carrying out the isomerization reaction may also include a tubular reactor. The tubular reactor may include an inner surface pre-heated to above a vaporizing temperature of the reactant composition. The reactant composition may be heated and vaporized before entering the tubular reactor, where the isomerization reaction is then conducted as the vaporized reactant composition is in contact with the heated inner surface of the tubular reactor. The reactors (either an electric heater reactor or a tubular reactor) may be first cleaned and flushed with an inert gas such as nitrogen. Suitable reactor materials include, for example, nickel and its alloys, including Hastelloy (for example, Hastelloy C276), Inconel (for example, Inconel 600), Incoloy, and Monel. Suitable reactor materials for a tubular reactor may also include stainless steel. The tubular reactor can have a single wall and be heated with an external furnace. Alternatively, the tubular reactor can be jacketed for heating with a circulating heat transfer medium, such as heating oil, thermal fluid, molten salt, etc.

The isomerization reaction may be conducted in a suitable reactor without use of a catalyst. The isomerization reaction may be conducted in a suitable reactor without use of irradiation, for example, ultraviolet radiation having a wavelength of from about 100 nm to about 400 nm, electromagnetic radiation having a wavelength of from about 380 nm to about 760 nm, infrared radiation having a wavelength of from about 780 nm to about 1 mm, and/or near-infrared radiation having a wavelength of from about 750 nm to about 2500 nm.

### Process Flow

FIGS. 1-3 include process flow diagrams of a process converting cis-1,2-difluoroethylene HFO-1132(Z) to trans-1,2-difluoroethylene (HFO-1132(E)). The process as shown in FIGS. 1a, 2a-2d, and 3a-3b may be used in isomerization reactions described below in Examples 1-9.

Referring to the once-through process flow diagram shown in FIG. 1a, feed stream (e.g., reactant composition) containing a mixture of HFO-1132(E) and HFO-1132(Z) or substantially pure HFO-1132(Z) (e.g., 99.9% 1132(Z)) is fed into a reactor 102 which is set at a desired reaction temperature. The pressure of reactor 102 may be about 14.7 psia or higher. The product passes through a caustic scrubber 106 (e.g., KOH scrubber), then through dryer tubes to remove water, and eventually is collected in a trap 104 which is cooled by dry ice. The reactor 102 may be an electric heater reactor (as shown) or a tubular reactor (not shown). Suitable tubular reactors for use as reactor 102 include, for example, a stainless steel reactor, or a Hastelloy reactor (e.g., Hastelloy C276). When the reactor 102 is an electric heater reactor, it is configured to heat at least a portion of the feed stream with an electric heating element. The feed stream may optionally go through one or more heat exchangers or pre-heaters (not shown) to be pre-heated and/or vaporized before being fed into the reactor 102.

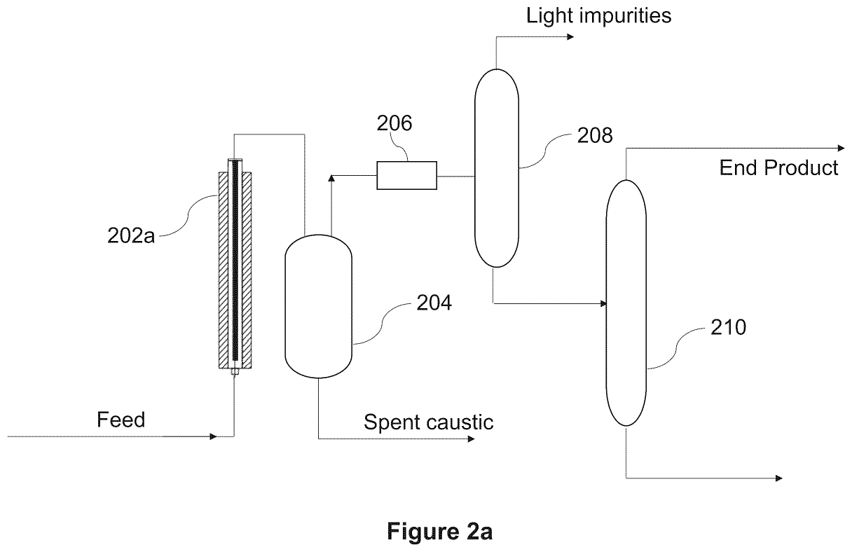

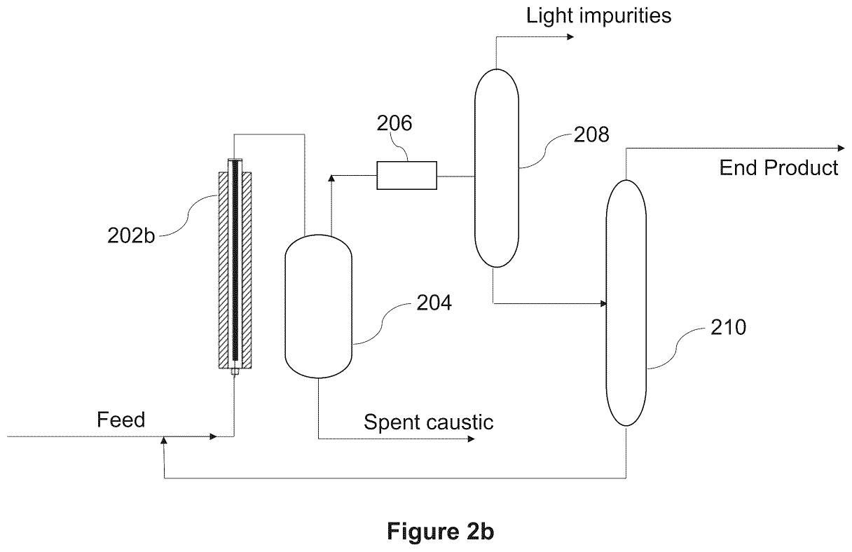

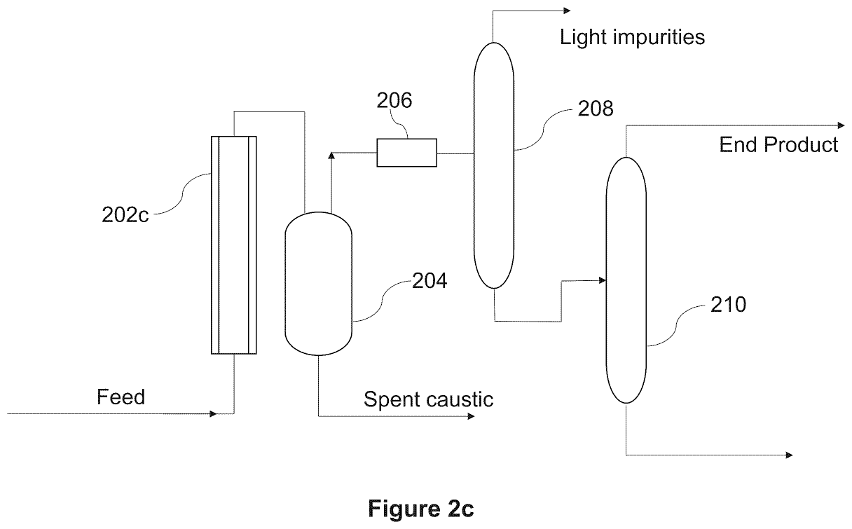

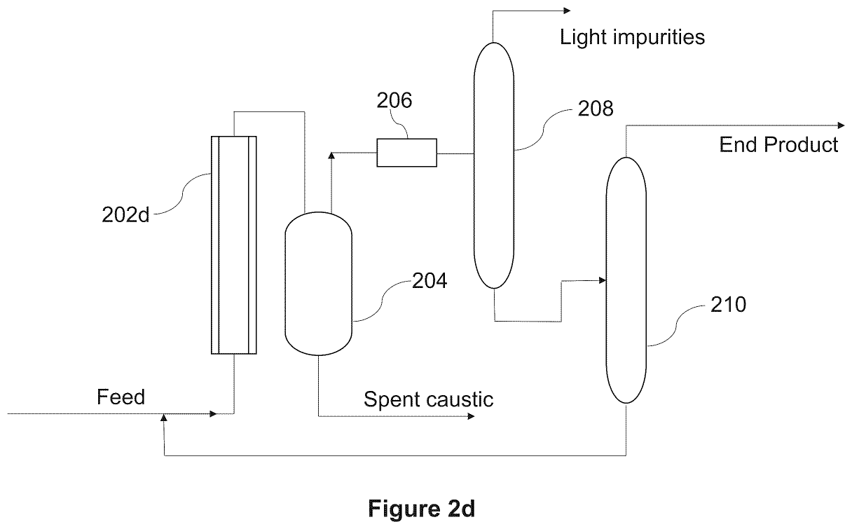

Referring to the process flow diagram shown in FIGS. 2a-2d, a feed stream (e.g., reactant composition) containing a mixture of HFO-1132(E) and HFO-1132(Z) or substantially pure HFO-1132(Z) (e.g., 99.9% 1132(Z)) is fed into an electric heater reactor 202a, 202b, or a tubular reactor 202c, 202d. The reactor 202a or 202b is an electric heater reactor with an immersion heating element (e.g., the electric heating element 12). The electric heating element of the electric heater reactor 202a or 202b is configured to heat at least a portion of the reactant composition during the reaction. The tubular reactor 202c or 202d can have a single wall and be heated with an external furnace or can be jacketed for heating with a circulating heat transfer medium, such as heating oil, thermal fluid, molten salt, etc. to heat at least a portion of the reactant composition during the reaction. The feed stream may optionally go through one or more heat exchangers or pre-heaters (not shown) to be pre-heated and/or vaporized before being fed into the electric heater reactor 202a, 202b or the tubular reactor 202c, 202d.

Since the boiling point of HFO-1132(Z) is −25° C., at least a portion of the feed stream containing a mixture of HFO-1132(E) and HFO-1132(Z) or substantially pure HFO-1132(Z) (e.g., 99.9% 1132(Z)) is vaporized at room temperature (e.g., 20-22° C.) before entering the reactor (e.g., reactor 102, 202a, 202b, 202c, 202d). The reactant stream may be pumped through the reactor 202a, 202b, 202c or 202d at a flow rate of about from about 10 kg/hr to about 12 kg/hr. The reactor 202a, 202b, 202c, or 202d may be pre-heated to a desired reaction temperature by electricity or other heat sources, such as molten salt, etc.

The effluent stream from the reactor 202a, 202b, 202c, or 202d may then flow through a heat exchanger (not shown) and further cool down by a cooler (not shown) before entering the caustic scrubber 204 to remove impurities (e.g., HF). The overhead stream from the scrubber 204 passes through a dryer 206 to remove moisture before entering the lights distillation column 208. Light impurities (e.g., vinyl fluoride, 1132a, ethyne, etc.) are removed from the overhead stream of the lights distillation column 208. The bottom stream from the lights distillation column 208 may flow through a cooler (not shown) and pressure regulating devices (e.g., pump, valve) before feeding into a separation column 210.

The bottom stream from the column 210 may optionally be recycled back to the frontend of the reactor and combined with the feed stream, for example as shown in FIGS. 2b and 2d. The bottom stream from column 210 may include, for example, a mixture of 1132(Z) and 1132(E) (e.g., 92.8% 1132(Z) and 7.2% 1132(E), or preferably 99.7% 1132(Z) and 0.3% 1132(E), or more preferably 99.9% 1132(Z) and 0.1% 1132(E)).

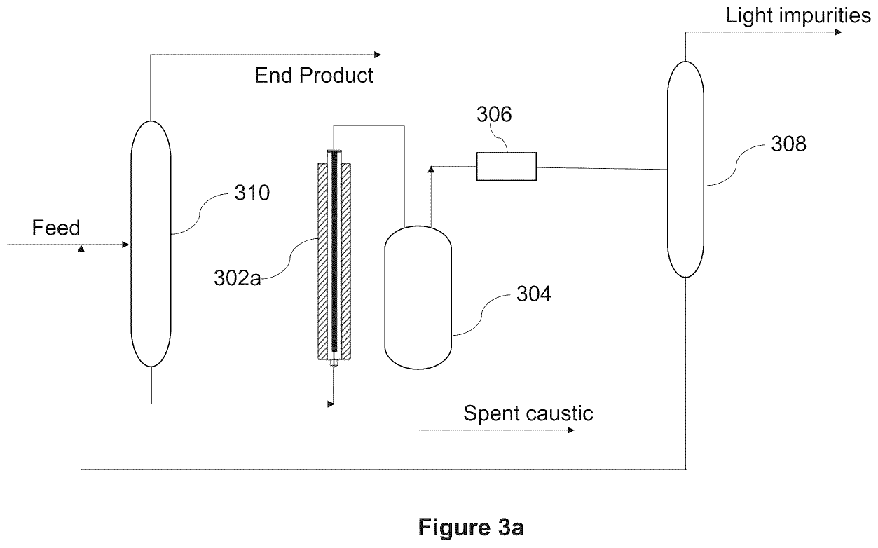

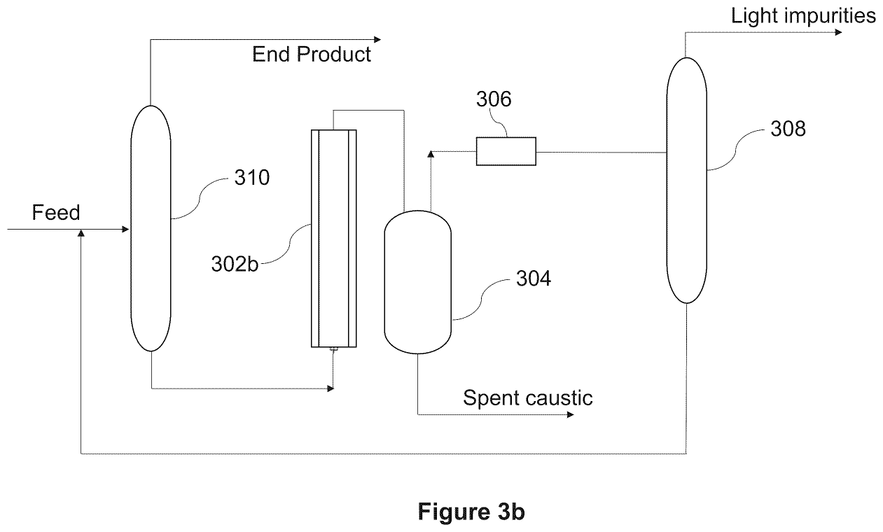

Referring to the process flow diagram shown in FIG. 3a and FIG. 3b, a feed stream containing a mixture of HFO-1132(E) and HFO-1132(Z) (e.g., 30% 1132(E) and 70% 1132(Z), or 90% 1132(Z) and 10% 1132(E)) is combined with a recycle stream from lights distillation column 308 to the separation column 310. The overhead stream of the column 310 is the product stream which has mainly 1132(E) (e.g., 99.9% pure) and the bottom stream is mainly 1132(Z) (e.g., 94.9% pure, preferably 99.7% pure, or more preferably 99.9% pure).

The bottom stream from column 310 may be fed into an electric heater reactor 302a with an immersion heating element configured to heat at least a portion of the bottom stream (e.g., HFO-1132(Z)) or a tubular reactor 302b heated with an external furnace or jacketed for heating with a circulating heat transfer medium. Optionally, the bottom stream from column 310 is heated and/or vaporized by flowing through a series of heat exchangers or pre-heaters (not shown) to a temperature above the boiling point of the feed stream before being fed into the electric heater reactor 302a or the tubular reactor 302b.

The reactor 302a or 302b may be pre-heated to a desired reaction temperature by electricity or other heat sources, such as molten salt, etc. The effluent stream from reactor 302a or 302b may then flow through a heat exchanger (not shown) to recover heat and further cooled down by a cooler (not shown) before entering a caustic scrubber 304 to remove impurities (e.g., HF). The overhead stream from the scrubber 304 flows through a dryer 306 to remove moisture and then goes into a lights distillation column 308. Light impurities (e.g., vinyl fluoride, 1132a, ethyne, etc.) are removed from the overhead stream of the lights distillation column 308. The bottom stream from the lights distillation column 308 is combined with the feed stream as the inlet stream for column 310.

The reactant stream containing HFO-1132(Z) may be fed into a reactor (e.g., reactor 102, 202a, 202b, 202c, 202d, 302a, 302b) to convert at least a portion of HFO-1132(Z) to HFO-1132(E) under conditions effective for the isomerization reaction of HFO-1132(Z) to HFO-1132(E) (i.e., Process 1). The electric reactor reactors 102, 202a, 202b, 302a may have a thermocouple on the end of the electric heating element for measuring the temperature. The feed stream may enter from the bottom of the reactor and flow out from the top of the reactor or vice versa.

### Reaction Condition—No Catalyst

The reaction may be a non-catalyzed isomerization reaction in a tubular reactor or an electric heater reactor. The reaction may be conducted without irradiation or UV light.

### Reaction Condition—Temperature

The temperature range for the isomerization reaction may be as low as about 325° C., about 350° C., about 400° C., about 450° C., or as high as about 500° C., about 550° C., about 600° C., about 650° C., or within any range encompassed by two of the foregoing values as endpoints, for example, from about 350° C. to about 600° C., preferably from about 400° C. to about 600° C., and more preferably from about 450° C. to about 550° C. Specific examples of additional suitable ranges are set forth below in Table 1.

TABLE 1
Reaction Temperature when using an Electric
Heater Reactor or a Tubular Reactor

| From (° C.) | To (° C.) |
|---|---|
| 325 | 650 |
| 350 | 650 |
| 350 | 600 |
| 350 | 550 |
| 350 | 500 |
| 400 | 650 |
| 400 | 600 |
| 400 | 550 |
| 400 | 500 |
| 450 | 650 |
| 450 | 600 |
| 450 | 550 |
| 450 | 500 |
| 350 | 400 |
| 400 | 450 |
| 450 | 500 |
| 500 | 550 |
| 550 | 600 |
| 600 | 650 |

### Reaction Condition-Pressure

The reaction may be conducted at atmospheric pressure, or above atmospheric pressure. The pressure may be as low as about 10 psia, 14.7 psia, 20 psia, 25 psia, or as high as about 35 psia, about 40 psia, about 45 psia, about 50 psia, about 55 psia, about 60 psia, about 100 psia, about 200 psia, or within any range encompassed by two of the foregoing values as endpoints, for example, from about 10 psia to 200 psia, preferably from about 14.7 psia to about 100 psia, and more preferably from about 14.7 psia to about 70 psia. Specific examples of additional suitable ranges are set forth below in Table 2.

TABLE 2
Reaction Pressure when using an Electric
Heater Reactor or a Tubular Reactor

| From (psia) | To (psia) |
|---|---|
| 10 | 70 |
| 10 | 100 |
| 10 | 120 |
| 10 | 150 |
| 10 | 180 |
| 10 | 200 |
| 14.7 | 70 |
| 14.7 | 100 |
| 14.7 | 120 |
| 14.7 | 150 |
| 14.7 | 180 |
| 14.7 | 200 |
| 10 | 14.7 |
| 14.7 | 40 |
| 40 | 50 |
| 50 | 70 |
| 70 | 90 |
| 90 | 100 |
| 100 | 120 |
| 120 | 140 |
| 140 | 160 |
| 160 | 180 |
| 180 | 200 |

### Reaction Condition-Residence Time

The residence time of the reactant stream in a reactor is the ratio of the reactor volume and feed rate (i.e., the rate at which the reactant stream is fed through a reactor). The residence time of a reactant stream in an electric reactor or a tubular reactor may be as little as about 0.1 second, about 1 second, about 2 seconds, about 5 seconds, about 10 seconds, about 15 seconds, about 20 seconds, or as long as about 25 seconds, about 30 seconds, about 40 seconds, about 50 seconds, about 60 seconds, about 80 seconds, about 120 seconds, about 140 seconds, about 150 seconds, or within any range encompassed by two of the foregoing values as endpoints, for example, from about 0.1 seconds to about 150 seconds, from about 0.1 second to about 140 seconds, from about 0.2 seconds to about 120 seconds, from about 0.3 seconds to about 60 seconds, from about 0.3 seconds to about 50 seconds, or about 0.5 seconds to about 40 seconds. Specific examples of additional suitable ranges are set forth below in Table 3.

TABLE 3
Residence Time of a Reactant Stream in an Electric
Heater Reactor or a Tubular Reactor

| From (seconds) | To (seconds) |
|---|---|
| 0.1 | 10 |
| 0.1 | 20 |
| 0.1 | 40 |
| 0.1 | 60 |
| 0.1 | 80 |
| 0.1 | 100 |
| 0.1 | 120 |
| 0.1 | 140 |
| 0.1 | 150 |
| 0.5 | 10 |
| 0.5 | 20 |
| 0.5 | 40 |
| 0.5 | 60 |
| 0.5 | 80 |
| 0.5 | 100 |
| 0.5 | 120 |
| 0.5 | 140 |
| 0.5 | 150 |
| 0.1 | 0.3 |
| 0.3 | 0.4 |
| 0.4 | 0.5 |
| 0.5 | 0.9 |
| 0.9 | 1 |
| 1 | 1.5 |
| 1.5 | 3 |
| 3 | 3.5 |
| 3.5 | 5 |
| 5 | 6 |
| 6 | 10 |
| 10 | 15 |
| 15 | 20 |
| 20 | 35 |
| 35 | 40 |
| 40 | 50 |
| 50 | 60 |
| 60 | 90 |
| 90 | 100 |
| 100 | 120 |
| 120 | 140 |
| 140 | 150 |

### Other Reaction Conditions

The rate at which the reactant stream is fed through a tubular reactor having an exemplary reactor volume of 31.4 mL, for example, may be as low as about 0.1 g/min, 0.5 g/min, about 1 g/min, about 1.5 g/min, about 3 g/min, about 5 g/min, or as high as about 10 g/min, about 15 g/min, about 20 g/min, about 25 g/min, about 30 g/min, about 35 g/min, about 40 g/min, or within any range encompassed by two of the foregoing values as endpoints, for example, from about 0.1 g/min to 40 g/min, preferably from about 0.5 g/min to about 40 g/min, and more preferably from about 0.5 g/min to about 35 g/min.

The reaction may also be conducted in an atmosphere substantially in the absence of oxygen. For example, the amount of oxygen present during the reaction may be less than 10 mol. %, less than 5 mol. %, or less than 1 mol. % based on a total amount of the reactants in the reactor, and for each of the foregoing, equal to or more than 0 mol. %, or within any range encompassed by two of the foregoing values as endpoints, based on total moles of the organic components of the composition.

The reaction may also be conducted substantially in the absence of water. For example, the amount of water present during the reaction may be less than 1 mol. %, less than 0.5 mol. %, or less than 0.05 mol. % based on a total amount of the reactants in the reactor, and for each of the foregoing, equal to or more than 0 mol. %, or within any range encompassed by two of the foregoing values as endpoints, based on total moles of the organic components of the composition.

A summary of reaction conditions including temperature, pressure, and residence time using an electric heater reactor or a tubular reactor as discussed above are summarized in Table 4 below.

TABLE 4
Various Reaction Conditions when using an Electric
Heater Reactor or a Tubular Reactor

| | | Residence Time |
|---|---|---|
| Temperature (° C.) | Pressure (psia) | (seconds) |
| 325-600 | 10-200 | 0.1-150 |
| 350-600 | 10-200 | 0.1-150 |
| 350-600 | 10-200 | 0.2-120 |
| 350-600 | 10-200 | 0.3-60 |
| 350-600 | 14.7-100 | 0.1-150 |
| 350-600 | 14.7-100 | 0.2-120 |
| 350-600 | 14.7-100 | 0.3-60 |
| 350-600 | 14.7-70 | 0.1-150 |
| 350-600 | 14.7-70 | 0.2-120 |
| 350-600 | 14.7-70 | 0.3-60 |
| 400-600 | 10-200 | 0.1-150 |
| 400-600 | 10-200 | 0.2-120 |
| 400-600 | 10-200 | 0.3-60 |
| 400-600 | 14.7-100 | 0.1-150 |
| 400-600 | 14.7-100 | 0.2-120 |
| 400-600 | 14.7-100 | 0.3-60 |
| 400-600 | 14.7-70 | 0.1-150 |
| 400-600 | 14.7-70 | 0.2-120 |
| 400-600 | 14.7-70 | 0.3-60 |
| 450-550 | 10-200 | 0.1-150 |
| 450-550 | 10-200 | 0.2-120 |
| 450-550 | 10-200 | 0.3-60 |
| 450-550 | 14.7-100 | 0.1-150 |
| 450-550 | 14.7-100 | 0.2-120 |
| 450-550 | 14.7-100 | 0.3-60 |
| 450-550 | 14.7-70 | 0.1-150 |
| 450-550 | 14.7-70 | 0.2-120 |
| 450-550 | 14.7-70 | 0.3-60 |

As demonstrated by the Examples herein, Process 1, using a non-catalytic integrated process, may form an end product including an amount of HFO-1132(E) of greater than about 20%, greater than about 30%, greater than about 40%, greater than about 50%, greater than about 60%, greater than about 70%, greater than about 80%, greater than about 85%, greater than about 90%, greater than about 95%, greater than about 98%, greater than about 99%, greater than about 99.9%, greater than about 99.99%, and for each of the foregoing, less than or equal to 100%, or within any range encompassed by two of the foregoing values as endpoints, based on total moles of the Products organic components of the end product composition. Specific examples of additional suitable ranges are set forth below in Table 5.

The end product in an integrated process may include a mixture of HFO-1132(Z) and HFO-1132(E), for example, from about 80% to about 99.99% of HFO-1132(E) and from about 0.01% to about 20% of 1132(Z), or from about 85% to about 99.99% of 1132(E) and from about 0.01% to about 15% 1132(Z), or from about 90% to about 99.99% of 1132(E) and from about 0.01% to about 10% 1132(Z), or from about 95% to about 99.99% of 1132(E) and from about 0.01% to about 5% 1132(Z), or from about 98% to about 99.99% of 1132(E) and from about 0.01% to about 2% 1132(Z), or within any range encompassing any two of these values as endpoints, based on total moles of the organic components of the end product composition.

TABLE 5
Amount of HFO-1132(E) in the End Product of an Integrated Process
using an Electric Heater Reactor or a Tubular Reactor

| From (%) | To (%) |
|---|---|
| 20 | ≤100 |
| 30 | ≤100 |
| 40 | ≤100 |
| 50 | ≤100 |
| 60 | ≤100 |
| 70 | ≤100 |
| 75 | ≤100 |
| 80 | ≤100 |
| 90 | ≤100 |
| 95 | ≤100 |
| 97 | ≤100 |
| 98 | ≤100 |
| 99 | ≤100 |
| 99.9 | ≤100 |

As also demonstrated by the Examples herein, Process 1, using a non-catalytic process in a once-through process, may achieve a conversion of HFO-1132(Z) to HFO-1132(E) of at least about 8%, at least about 16%, at least about 20%, at least about 25%, at least about 30%, at least about 35% or greater, and for each of the foregoing, less than or equal to 40%, or within any range encompassed by two of the foregoing values as endpoints, based on total moles of the organic components of the once-through product composition. Specific examples of additional suitable ranges are set forth below in Table 6.

TABLE 6
Conversion of HFO-1132(Z) in a Once-through Process
using an Electric Heater Reactor or a Tubular Reactor

| From (%) | To (%) |
|---|---|
| 8 | 40 |
| 10 | 40 |
| 15 | 40 |
| 16 | 40 |
| 20 | 40 |
| 25 | 40 |
| 30 | 40 |
| 35 | 40 |
| 8 | 10 |
| 10 | 15 |
| 15 | 16 |
| 16 | 20 |
| 20 | 25 |
| 25 | 30 |
| 30 | 35 |

After a feed stream including substantially pure 1132(Z) (e.g., 99.99% of 1132(Z)) goes through an electric heater reactor under Process 1 in a once-through process, the amount of 1132 (E) in the once-through product composition may be at least about 8%, at least about 16%, at least about 20%, at least about 25%, at least about 30%, at least about 35% or greater, and for each of the foregoing, less than or equal to 40%, or within any range encompassed by two of the foregoing values as endpoints, based on total moles of the organic components of the once-through product composition. Specific examples of additional suitable ranges are set forth below in Table 7.

TABLE 7
Amount of HFO-1132(E) in a Once-through Product Composition using
an Electric Heater Reactor or a Tubular Reactor in Process 1

| From (%) | To (%) |
|---|---|
| 8 | 40 |
| 10 | 40 |
| 15 | 40 |
| 16 | 40 |
| 20 | 40 |
| 25 | 40 |
| 30 | 40 |
| 35 | 40 |
| 8 | 10 |
| 10 | 15 |
| 15 | 16 |
| 16 | 20 |
| 20 | 25 |
| 25 | 30 |
| 30 | 35 |

Process 1 may be conducted in an electric heater reactor without use of a catalyst, at a temperature of from about 325° C. to about 425° C., or at a temperature of from about 350° C. to about 400° C., under a pressure of from about 14.7 psia to about 50 psia, and with a residence time of from about 10 seconds to about 40 seconds. The amount of 1132 (E) in the once-through product composition after a single pass reaction may be from about 8% to about 40%, based on total moles of the organic components of the product composition. The amount of 1132 (E) in the end product mixture in an integrated process using the same reactor and reaction conditions may be from about 99.9% to about 100%, based on total moles of the organic components of the end product.

Process 1 may be conducted in an electric heater reactor without use of a catalyst, at a temperature of from about 425° C. to about 475° C., or at a temperature of about 450° C., under a pressure of from about 14.7 psia to about 50 psia, and with a residence time of from about 3 seconds to about 15 seconds. The amount of 1132 (E) in the once-through product composition after a single pass reaction may be from about 33% to about 40%, based on total moles of the organic components of the product composition. The amount of 1132 (E) in the end product mixture in an integrated process using the same reactor and reaction conditions may be from about 99.9% to about 100%, based on total moles of the organic components of the end product.

Process 1 may be conducted in an electric heater reactor without use of a catalyst, at a temperature of from about 475° C. to about 600° C., or preferably from about 500° C. to about 600° C., under a pressure of about 14.7 psia, and with a residence time of from about 0.1 second to about 4 seconds. The amount of 1132 (E) in the once-through product composition after a single pass reaction may be from about 34% to about 40%, or from about 35% to about 100%, based on total moles of the organic components of the product mixture. The amount of 1132 (E) in the end product mixture in an integrated process using the same reactor and reaction conditions may be from about 99.9% to about 100%, based on total moles of the organic components of the end product.

### EXAMPLES

### Example 1—Isomerization of 1132(Z) to 1132(E) (Single Pass Through an Electric Heater Reactor)

The process may be carried out as described above and shown in FIG. 1a under conditions included in Table 8 below. The pressure of the reactor is 14.7 psia or higher. The outer diameter “d” of the electric heating element (e.g., electric heating element 12 of the electric heater reactor 10 as shown in FIG. 1b) is 0.495 inches which is inserted inside an Inconel reactor tube (e.g., reactor tube 16 as shown in FIG. 1b). The reactor tube has an internal diameter “D” of 0.62 inches and a reactor tube external diameter of 0.75 inches. The length of the heating element “L” is 17.5 inches. The reaction is conducted in the gap (with the volume of 31.4 ml) between the internal heating element and external tube (e.g., space 18 as shown in FIG. 1b). At least two thermocouples are inserted from the side into the empty gap to read the operating temperature during the isomerization reaction.

The reaction is conducted at a temperature of from about 350° C. to about 650° C., or preferably from about 400° C. to about 500° C. The conversion of 1132 (Z) to 1132(E) after a single pass through the reactor is from about 1% to about 37%, based on total moles of the organic components of the product composition.

TABLE 8
Process conditions and product compositions for
a single pass through an electric heater reactor

| Electric | | | | | |
|---|---|---|---|---|---|
| heater | | | Feed | Residence time | |
| temperature | Pressure | Feed stream | rate | for Process 1 | Product |
| (° C.) | (psia) | composition | (g/hr) | (sec) | composition |
| 350 | 14.7 | 99.9% | 1.0 | 142.0 | 82.3% 1132(Z), |
| | | 1132(Z) | | | 17.6% 1132(E), |
| | | | | | 0.1% others |
| 350 | 35.0 | 99.9% | 2.5 | 135.7 | 82.9% 1132(Z), |
| | | 1132(Z) | | | 17.0% 1132(E), |
| | | | | | 0.1% others |
| 350 | 50.0 | 99.9% | 5.0 | 97.2 | 86.5% 1132(Z), |
| | | 1132(Z) | | | 13.4% 1132(E), |
| | | | | | 0.1% others |
| 400 | 14.7 | 99.9% | 2.5 | 52.6 | 70.0% 1132(Z), |
| | | 1132(Z) | | | 29.8% 1132(E), |
| | | | | | 0.2% others |
| 400 | 35.0 | 99.9% | 5.0 | 62.7 | 68.8% 1132(Z), |
| | | 1132(Z) | | | 31.0% 1132(E), |
| | | | | | 0.2% others |
| 400 | 50.0 | 99.9% | 10.0 | 44.9 | 71.4% 1132(Z), |
| | | 1132(Z) | | | 28.4% 1132(E), |
| | | | | | 0.2% others |
| 450 | 14.7 | 99.9% | 15.0 | 8.1 | 70.0% 1132(Z), |
| | | 1132(Z) | | | 29.8% 1132(E), |
| | | | | | 0.2% others |
| 450 | 35.0 | 99.9% | 20.0 | 14.6 | 66.5% 1132(Z), |
| | | 1132(Z) | | | 33.3% 1132(E), |
| | | | | | 0.2% others |
| 450 | 50.0 | 99.9% | 30.0 | 13.9 | 66.7% 1132(Z), |
| | | 1132(Z) | | | 33.1% 1132(E), |
| | | | | | 0.2% others |
| 500 | 14.7 | 99.9% | 50.0 | 2.3 | 67.1% 1132(Z), |
| | | 1132(Z) | | | 32.4% 1132(E), |
| | | | | | 0.5% others |
| 550 | 14.7 | 99.9% | 70.0 | 1.5 | 64.0% 1132 (Z), |
| | | 1132(Z) | | | 35.5% 1132(E), |
| | | | | | 0.5% others |
| 600 | 14.7 | 99.9% | 70.0 | 1.4 | 62.2% 1132(Z), |
| | | 1132(Z) | | | 36.7% 1132(E), |
| | | | | | 1.1% others |
| 650 | 14.7 | 99.9% | 80.0 | 1.2 | 61.5% 1132(Z), |
| | | 1132(Z) | | | 36.0% 1132(E), |
| | | | | | 2.5% others |

### Example 2—Isomerization of 1132(Z) to 1132(E) (Electric Heater Reactor without Recycle Stream)

The process may be carried out as described above and shown in FIG. 2a without an optional recycle stream as part of the initial feed stream. A feed stream including 99.0% 1132(Z) and 1.0% 1132(E) (flow rate: 22 g/hr) is fed into an electric heater reactor 202a configured to heat the stream. The dimensions of the heating element and reactor tube of the electric heater reactor are the same as described above in Example 1. The reaction is performed at 450° C. and 50.0 psia. The residence time of the process stream in the reactor is about 19 seconds. The product flows through a caustic scrubber 204 and a dryer 206. The light impurities are separated from the lights distillation column 208. The lights distillation column 208 has 20-30 stages and an overhead pressure of 55-65 psig. The bottom stream from the lights distillation column 208 flows through a cooler (not shown) and pressure regulating devices (e.g., pump, valve) to a bubble point at P=65 psig before feeding into a separation column 210 to separate 1132Z and 1132E. The separation column 210 has 20 stages with an overhead pressure of about 55-60 psig.

The purity in the end product stream is 99.9% 1132(E), based on total moles of organic components of the end product composition. The overall yield of 1132(E) is about 32%, based on the total amount of initial reactant in the feed stream.

### Example 3—Isomerization of 1132(Z) to 1132(E) (Electric Heater Reactor with Recycle Stream)

The process may be carried out as described above and shown in FIG. 2b. A fresh feed stream of 99.0% 1132(Z) and 1.0% 1132(E) (flow rate: 22 g/h) is combined with a recycle stream from the bottom of the separation column 210 and fed into an electric heater reactor 202b configured to heat the stream with an electric heating element. The dimensions of the heating element and reactor tube of the electric heater reactor are the same as described above in Example 1. The reaction is performed at 500° C. and 50 psia. The residence time of the process stream in the reactor is about 6 seconds. The reactor effluent flows through a caustic scrubber 204 and a dryer 206. The light impurities are separated in a lights distillation column 208. The bottom stream of the lights distillation column 208 flows into a separation column 210. The bottom stream from the lights distillation column 208 contains predominantly 1132(Z) is recycled back to the frontend of the reactor 202b after flowing through the separation column 210.

The overhead stream from the column 210 is the end product, which has 99.9% 1132(E) based on total moles of organic components of the end product composition. The overall yield of 1132(E) is greater than 95%, based on the total amount of initial reactant in the feed stream.

### Example 4—Isomerization of 1132(Z) to 1132(E) (Electric Heater Reactor with Recycle Stream with High 1132 (E) in Feed)

The process may be carried out as described above and shown in FIG. 3a. A feed stream of 30.0% 1132(E) and 70.0% 1132(Z) (flow rate: 22 g/h) is combined with a recycle stream from the lights distillation column 308 and is fed into column 310 to generate an end product stream and a combined feed stream which is fed to the electric heater reactor 302a. The dimensions of the heating element and reactor tube of the electric heater reactor are the same as described above in Example 1. The reaction is performed at 500° C. and 50 psia. The residence time of the process stream in the reactor is about 8 seconds. The reactor effluent stream flows through a scrubber 304 and a dryer 306. The light impurities are separated using a lights distillation column 308. The bottom stream is recycled back to the front end of the column 310.

The overhead stream from the column 310 is the end product, which has 99.9% 1132(E), based on total moles of organic components of the end product composition. The overall yield of 1132E is greater than 95%, based on the total amount of initial reactant in the feed stream.

### Example 5—Isomerization of 1132(Z) to 1132(E) (Electric Heater Reactor with Recycle Stream with a Mixture of 1132(E) and 1132(Z) in Feed)

The process may be carried out as described above and shown in FIG. 3a. A feed stream of 10.0% 1132(E) and 90.0% 1132(Z) (flow rate: 22 g/h) is combined with a recycle stream from the lights distillation column 308 and is fed into the column 310 to generate an end product stream and a combined feed stream which is fed to the electric heater reactor 302a. The dimensions of the heating element and reactor tube of the electric heater reactor 302a are the same as described above in Example 1. The reaction is performed at 500° C. and 50 psia. The residence time of the process stream in the reactor is about 7 seconds. The reactor effluent stream flows through a scrubber 304 and a dryer 306. The light impurities are separated using a lights distillation column 308. The reactor effluent is pressurized to the bubble point at P=65 psig before feeding into a separation column 310. The column 310 has 20 stages with an overhead pressure set at P=55-60 psig. The bottom stream is recycled back to the front end of the column 310.

The overhead stream from the column 310 is the end product, which has 99.9% 1132(E), based on total moles of organic components of the end product composition. The overall yield is greater than 95%, based on the total amount of initial reactant in the feed stream.

### Example 6—Isomerization of 1132(Z) to 1132(E) (Single Pass Through a Tubular Reactor)

The process may be carried out as described above and shown in FIG. 1a using a tubular reactor (not shown) instead of an electric reactor 102. The tubular reactor has an internal diameter of about 0.5″ OD (0.43″ ID) and a length of about 13.2″. The pressure of the reactor is 14.7 psia or higher. The reaction conditions are the same as described above in Example 1. A stream of 99.9% 1132(Z) and 0.1% 1132(E) passes through a series of heat exchangers to be heated and vaporized to a temperature of above 400° C. before being fed through a tubular reactor. The tubular reactor is pre-heated to a temperature of above 400° C. The conversion of 1132 (Z) to 1132(E) after a single pass through the tubular reactor is acceptable.

### Example 7—Isomerization of 1132(Z) to 1132(E) (Tubular Reactor without Recycle Stream)

The process may be carried out as described above and shown in FIG. 2c without the optional recycle stream as part of the initial feed stream. The reaction condition and tubular reactor dimensions are the same as described above in Example 6. A stream of 99.0% 1132(Z) and 1.0% 1132(E) (flow rate: 22 g/h) is vaporized first, then fed into a pre-heated tubular reactor. The reaction is performed at 500° C. and 19.0 psia. The residence time of the process stream in the reactor is about 7 sec.

The purity of 1132(E) in the product stream and overall yield of 1132(E) are acceptable.

### Example 8—Isomerization of 1132(Z) to 1132(E) (Tubular Reactor with Recycle Stream)

The process may be carried out as described above and shown in FIG. 2d. A feed stream of 99.0% 1132(Z) and 1.0% 1132(E) (flow rate: 22 g/h) is combined with a recycle stream from the bottom of the separation column 210, vaporized first, then fed into a pre-heated tubular reactor 202d. The reaction condition and tubular reactor dimensions are the same as described above in Example 6. The reaction is performed at 500° C. and 50 psia. The residence time of the process stream in the reactor is about 6 sec. The bottom stream from the separation column 208 flows into a separation column 210. The overhead stream from column 210 is the end product and the bottom stream is recycled back to the frontend of the reactor 202b.

The purity of 1132(E) in the product stream and overall yield of 1132(E) are acceptable.

### Example 9—Isomerization of 1132(Z) to 1132(E) (Tubular Reactor with Recycle Stream with High 1132 (E) in Feed)

The process may be carried out as described above and shown in FIG. 3b. The reaction condition and tubular reactor dimensions are the same as described above in Example 6. A feed stream of 10.0% 1132(E) and 90.0% 1132(Z) (flow rate: 22 g/h) is combined with a recycle stream from the lights distillation column 308. The lights distillation column 308 has 20-30 stages and an overhead pressure of 55-65 psig. The bottom stream from the separation column 310 is vaporized first, then fed into a pre-heated tubular reactor 302b. The reaction is performed at 500° C. and 50 psia. The residence time of the process stream in the reactor is about 8 sec.

The purity of 1132(E) in the product stream and overall yield of 1132(E) are acceptable.

### Example 10—Isomerization of 1132(Z) to 1132(E) (Single Pass Through an Electric Heater Reactor Using a Mixture of 1132(Z) and 1132(E) as Feed Stream)

The process may be carried out as described above and shown in FIG. 1a under conditions included in Table 9 below. The pressure of the reactor is 14.7 psia or higher. The outer diameter “d” of the electric heating element (e.g., electric heating element 12 of the electric heater reactor 10 as shown in FIG. 1b) is 0.495 inches which is inserted inside an Inconel reactor tube (e.g., reactor tube 16 as shown in FIG. 1b). The reactor tube has an internal diameter “D” of 0.62 inches and a reactor tube external diameter of 0.75 inches. The length of the heating element “L” is 17.5 inches. The reaction is conducted in the gap (with the volume of 31.4 ml) between the internal heating element and external tube (e.g., space 18 as shown in FIG. 1b). At least two thermocouples are inserted from the side into the empty gap to read the operating temperature during the isomerization reaction.

The reaction is conducted at a temperature of from about 350° C. to about 650° C., or preferably from about 400° C. to about 500° C. The conversion of 1132 (Z) to 1132(E) after a single pass through the reactor is from about 10% to about 31%, based on total moles of the organic components of the product composition.

TABLE 9
Process conditions and product composition for a single pass through an electric
heater reactor using a mixture of 1132(Z) and 1132(E) as feed stream

| Electric | | | | | |
|---|---|---|---|---|---|
| heater | | | Feed | Residence time | |
| temperature | Pressure | Feed stream | rate | for Process 1 | Product |
| (° C.) | (psia) | composition | (g/hr) | (sec) | composition |
| 350 | 14.7 | 90% 1132(Z) | 1.0 | 142.0 | 77.7% 1132(Z), |
| | | 10% 1132(E) | | | 22.2% 1132(E), |
| | | | | | 0.1% others |
| 350 | 35.0 | 90% 1132(Z) | 2.5 | 135.7 | 78.2% 1132(Z), |
| | | 10% 1132(E) | | | 21.7% 1132(E), |
| | | | | | 0.1% others |
| 350 | 50.0 | 90% 1132(Z) | 5.0 | 97.2 | 80.7% 1132(Z), |
| | | 10% 1132(E) | | | 19.2% 1132(E), |
| | | | | | 0.1% others |
| 400 | 14.7 | 90% 1132(Z) | 2.5 | 52.6 | 69.0% 1132(Z), |
| | | 10% 1132(E) | | | 30.8% 1132(E), |
| | | | | | 0.2% others |
| 400 | 35.0 | 90% 1132(Z) | 5.0 | 62.7 | 68.2% 1132(Z), |
| | | 10% 1132(E) | | | 31.6% 1132(E), |
| | | | | | 0.2% others |
| 400 | 50.0 | 90% 1132(Z) | 10.0 | 44.9 | 70.0% 1132(Z), |
| | | 10% 1132(E) | | | 29.8% 1132(E), |
| | | | | | 0.2% others |
| 450 | 14.7 | 90% 1132(Z) | 15.0 | 8.1 | 68.8% 1132(Z), |
| | | 10% 1132(E) | | | 31.0% 1132(E), |
| | | | | | 0.2% others |
| 450 | 35.0 | 90% 1132(Z) | 20.0 | 14.6 | 66.3% 1132(Z), |
| | | 10% 1132(E) | | | 33.5% 1132(E), |
| | | | | | 0.2% others |
| 450 | 50.0 | 90% 1132(Z) | 30.0 | 13.9 | 66.4% 1132(Z), |
| | | 10% 1132(E) | | | 33.4% 1132(E), |
| | | | | | 0.2% others |
| 500 | 14.7 | 90% 1132(Z) | 50.0 | 2.3 | 66.3% 1132(Z), |
| | | 10% 1132(E) | | | 33.2% 1132(E), |
| | | | | | 0.5% others |
| 550 | 14.7 | 90% 1132(Z) | 70.0 | 1.5 | 63.8% 1132 (Z), |
| | | 10% 1132(E) | | | 35.7% 1132(E), |
| | | | | | 0.5% others |
| 600 | 14.7 | 90% 1132(Z) | 70.0 | 1.4 | 62.8% 1132(Z), |
| | | 10% 1132(E) | | | 36.1% 1132(E), |
| | | | | | 1.1% others |
| 650 | 14.7 | 90% 1132(Z) | 80.0 | 1.2 | 61.5% 1132(Z), |
| | | 10% 1132(E) | | | 36.0% 1132(E), |
| | | | | | 2.5% others |

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### Aspects:

Aspect 1 is a method for producing trans-1,2-difluoroethylene (HFO-1132(E)), comprising: providing a reactant composition comprising cis-1,2-difluoroethylene (HFO-1132(Z)); and isomerizing the HFO-1132(Z) in an electric heater reactor at a temperature of from about 350° C. to about 600° C. to produce a product composition comprising HFO-1132(E).

Aspect 2 is the method of Aspect 1, wherein the electric heater reactor comprises an electric heating element configured to heat the reactant composition.

Aspect 3 is the method of Aspect 2, wherein the isomerizing step is conducted where the reactant composition is in contact with the electric heating element.

Aspect 4 is the method of any one of Aspects 1-3, wherein the isomerizing step is conducted without use of a catalyst.

Aspect 5 is the method of any one of Aspects 1-4, wherein the isomerizing step is conducted without irradiation.

Aspect 6 is the method of any one of Aspect 1-5, wherein the isomerizing step is conducted at a pressure of from about 14.7 psia to about 200 psia.

Aspect 7 is the method of any one of Aspects 1-6, wherein the isomerizing step is conducted with a residence time of from about 0.1 second to about 150 seconds.

Aspect 8 is the method of any one of Aspects 1-7, wherein the isomerizing step is conducted at a temperature of from about 325° C. to about 425° C. with a residence time of from about 10 seconds to about 150 seconds.

Aspect 9 is the method of any one of Aspects 1-7, wherein the isomerizing step is conducted at a temperature of from about 425° C. to about 475° C. with a residence time of from about 3 seconds to about 140 seconds.

Aspect 10 is the method of Aspect 7, wherein the isomerizing step is conducted at a temperature of from about 475° C. to about 600° C. with a residence time of from about 0.1 second to about 130 seconds.

Aspect 11 is the method of any one of Aspects 1-7, wherein the isomerizing step is conducted at a temperature of from about 350° C. to about 400° C., a pressure of from about 35 psia to about 200 psia, and with a residence time of from about 10 seconds to about 150 seconds; wherein the product composition comprises equal to or greater than 30% 1132(E) and equal to or less than 70% 1132(Z) after a single pass through the electric heater reactor, based on total amount of organic components of the product composition.

Aspect 12 is the method of any one of Aspects 1-7, wherein the isomerizing step is conducted at a temperature of about 450° C., a pressure of from about 14.7 psia to about 200 psia, and with a residence time of from about 3 seconds to about 140 seconds; wherein the product composition comprises equal to or greater than 33.5% 1132(E) and equal to or less than 66.2% 1132(Z) after a single pass through the electric heater reactor, based on total amount of organic components of the product composition.

Aspect 13 is the method of any one of Aspects 1-7, wherein the isomerizing step is conducted at a temperature of from about 500° C. to about 600° C., a pressure of about 14.7 psia, and with a residence time of from about 0.1 second to about 130 seconds; wherein the product composition comprises at least 35.5% 1132(E) and at most 64% 1132(Z) after a single pass through the electric heater reactor, based on total amount of the product composition.

Aspect 14 is an integrated system for the production of HFO-1132(E), the system comprising: an electric heater reactor comprising an electric heating element configured to maintain a reactor temperature from about 350° C. to about 600° C. and to heat at least part of a reactant composition to produce a product composition comprising HFO-1132(E); and a separator fluidly coupled to an outlet of the electric heater reactor, the separator configured to produce a concentrated product composition having a concentration of HFO-1132(E) that is greater than a concentration of HFO-1132(Z) in the reactant composition.

Aspect 15 is the system of Aspect 14, wherein the separator produces a recycle stream recycled back to the electric heater reactor.

Aspect 16 is a method for producing trans-1,2-difluoroethylene (HFO-1132(E)), comprising: providing a reactant composition comprising cis-1,2-difluoroethylene (HFO-1132(Z)); and isomerizing the HFO-1132(Z) in a tubular reactor at a temperature of from about 350° C. to about 600° C. to produce a product composition comprising HFO-1132(E).

Aspect 17 is the method of Aspect 16, wherein the tubular reactor comprises a surface heated to above a vaporizing temperature of the reactant composition; wherein the reactant composition is vaporized before entering the tubular reactor; wherein the isomerizing step is conducted where the vaporized reactant composition is in contact with the heated surface of the tubular reactor.

Aspect 18 is the method of Aspect 16 or 17, wherein the isomerizing step is conducted without use of a catalyst or irradiation.

Aspect 19 is the method of any one of Aspects 16-18, wherein the isomerizing step is conducted at a pressure of from about 14.7 psia to about 200 psia; wherein the isomerizing step is conducted with a residence time of from about 0.1 second to about 150 seconds.

Aspect 20 is the method of any one of Aspects 16-19, wherein the product composition comprises at least 30 wt. % of HFO-1132(E) based on total amount of the product composition.

1. A method for producing trans-1,2-difluoroethylene (HFO-1132(E)), comprising:
providing a reactant composition comprising cis-1,2-difluoroethylene (HFO-1132(Z)); and
isomerizing the HFO-1132(Z) in an electric heater reactor at a temperature of from about 350° C. to about 600° C. to produce a product composition comprising HFO-1132(E).

2. The method of claim 1, wherein the electric heater reactor comprises an electric heating element configured to heat the reactant composition.

3. The method of claim 2, wherein the isomerizing step is conducted where the reactant composition is in contact with the electric heating element.

4. The method of claim 1, wherein the isomerizing step is conducted without use of a catalyst.

5. The method of claim 1, wherein the isomerizing step is conducted without irradiation.

6. The method of claim 1, wherein the isomerizing step is conducted at a pressure of from about 14.7 psia to about 200 psia.

7. The method of claim 1, wherein the isomerizing step is conducted with a residence time of from about 0.1 second to about 150 seconds.

8. The method of claim 1, wherein the isomerizing step is conducted at a temperature of from about 325° C. to about 425° C. with a residence time of from about 10 seconds to about 150 seconds.

9. The method of claim 1, wherein the isomerizing step is conducted at a temperature of from about 425° C. to about 475° C. with a residence time of from about 3 seconds to about 140 seconds.

10. The method of claim 1, wherein the isomerizing step is conducted at a temperature of from about 475° C. to about 600° C. with a residence time of from about 0.1 second to about 130 seconds.

11. The method of claim 1, wherein the isomerizing step is conducted at a temperature of from about 350° C. to about 400° C., a pressure of from about 35 psia to about 200 psia, and with a residence time of from about 10 seconds to about 150 seconds;
wherein the product composition comprises equal to or greater than 30% 1132(E) and equal to or less than 70% 1132(Z) after a single pass through the electric heater reactor, based on total amount of organic components of the product composition.

12. The method of claim 1, wherein the isomerizing step is conducted at a temperature of about 450° C., a pressure of from about 14.7 psia to about 200 psia, and with a residence time of from about 3 seconds to about 140 seconds;
wherein the product composition comprises equal to or greater than 33.5% 1132(E) and equal to or less than 66.2% 1132(Z) after a single pass through the electric heater reactor, based on total amount of organic components of the product composition.

13. The method of claim 1, wherein the isomerizing step is conducted at a temperature of from about 500° C. to about 600° C., a pressure of about 14.7 psia, and with a residence time of from about 0.1 second to about 130 seconds;
wherein the product composition comprises at least 35.5% 1132(E) and at most 64% 1132(Z) after a single pass through the electric heater reactor, based on total amount of organic components of the product composition.

14. An integrated system for the production of HFO-1132(E), the system comprising:
an electric heater reactor comprising an electric heating element configured to maintain a reactor temperature from about 350° C. to about 600° C. and to heat at least part of a reactant composition to produce a product composition comprising HFO-1132(E); and
a separator fluidly coupled to an outlet of the electric heater reactor and configured to produce a concentrated product composition having a concentration of HFO-1132(E) greater than a concentration of HFO-1132(Z) in the reactant composition.

15. The system of claim 14, wherein the separator produces a recycle stream recycled back to the electric heater reactor.

16. A method for producing trans-1,2-difluoroethylene (HFO-1132(E)), comprising:
providing a reactant composition comprising cis-1,2-difluoroethylene (HFO-1132(Z)); and
isomerizing the HFO-1132(Z) in a tubular reactor at a temperature of from about 350° C. to about 600° C. to produce a product composition comprising HFO-1132(E).

17. The method of claim 16, wherein the tubular reactor comprises a surface heated to above a vaporizing temperature of the reactant composition;
wherein the reactant composition is vaporized before entering the tubular reactor;
wherein the isomerizing step is conducted where the vaporized reactant composition is in contact with the heated surface of the tubular reactor.

18. The method of claim 16, wherein the isomerizing step is conducted without use of a catalyst or irradiation.

19. The method of claim 16, wherein the isomerizing step is conducted at a pressure of from about 14.7 psia to about 200 psia;
wherein the isomerizing step is conducted with a residence time of from about 0.1 second to about 150 seconds.

20. The method of claim 16, wherein the product composition comprises at least 30 wt. % of HFO-1132(E) based on total amount of the product composition.
